(19) United States
(12) Patent Application Publication
(10) Pub. No.: US 2024/0091115 A1
(43) Pub. Date: Mar. 21, 2024

(54) Oral Care Compositions and Methods of Use

(71) Applicant: Colgate-Palmolive Company, New York, NY (US)

(72) Inventors: Payal ARORA, Lebanon, NJ (US); Shashank POTNIS, North Brunswick, NJ (US); Melissa MARTINETTI, Bridgewater, NJ (US); Ariel HASKEL, East Brunswick, NJ (US); Yun XU, Langhorne, PA (US); David SURIANO, Edison, NJ (US)

(73) Assignee: Colgate-Palmolive Company, New York, NY (US)

(21) Appl. No.: 17/766,973

(22) PCT Filed: Oct. 6, 2020
(86) PCT No.: PCT/US2020/070619

§ 371 Date: Apr. 6, 2022

Related U.S. Application Data
(60) Provisional application No. 62/911,645, filed on Oct. 7, 2019.

(51) Int. Cl. A61K 8/34; A61K 8/21; A61K 8/44; A61K 8/58; A61Q 11/00

(52) U.S. Cl. CPC A61K 8/347; A61K 8/21; A61K 8/442; A61K 8/58; A61Q 11/00

(57) ABSTRACT

The present disclosure relates to oral care compositions comprising amino acids and a cannabinoid. Methods of making and use are further provided.

### FIELD

This invention relates to oral care compositions, e.g., a dentifrice in the form of a gel, paste, powder or mouthwash. In some embodiments, the oral care compositions of the present disclosure comprise an amino acid, and a cannabinoid, as well as to methods of making and using such compositions.

### BACKGROUND

Cannabinoids are a class of diverse chemical compounds that act on cannabinoid receptors in cells that alter neurotransmitter release in the brain. There are at least 113 different cannabinoids isolated from Cannabis, exhibiting varied effects. While delta-9-tetrahydrocannabinol (THC) is the major active ingredient of Cannabis extracts, cannabidiol makes up about 40% of Cannabis extracts and has been studied for many different uses. It is known that cannabidiol lacks the psychoactive effects seen in many of the other cannabinoids including delta-9-tetrahydrocannabinol (THC).

Unchecked bacterial growth in the oral cavity can lead to a number of adverse conditions. For example, gingivitis is an inflammation of the gums, and is one of the most common disorders of the oral cavity. It is ordinarily caused by bacterial accumulations on the surface of the teeth, which may be in the form of plaque. Gingivitis results in a number of unpleasant symptoms including inflamed gums that are painful or sensitive, halitosis, and bleeding from the gums while brushing or flossing. Other common disorders of the mouth include abscesses and cold sores, which also involve inflammation and are painful to those afflicted. In order to treat bacterial-related disorders of the mouth, toothpastes are routinely manufactured with antibacterial agents.

Without being bound by theory, it is believed that cannabinoids such as CBD, with its antibacterial and anti-inflammatory properties, are well-suited to treat a variety of oral diseases and disorders.

Arginine and other basic amino acids have been proposed for use in oral care and are believed to have benefits in combating cavity formation and tooth sensitivity. Combining these basic amino acids with minerals having oral care benefits, e.g., fluoride and calcium, to form an oral care product having acceptable long-term stability, however, has proven challenging. In particular, the basic amino acid may raise the pH and facilitate dissociation of calcium ions that can react with fluoride ions to form an insoluble precipitate. Moreover, the higher pH has the potential to cause irritation. At neutral pH or acidic pH, however, a system utilizing arginine bicarbonate (which the art teaches is preferred) may release carbon dioxide, leading to bloating and bursting of the containers. Moreover, it might be expected that lowering the pH to neutral or acidic conditions would reduce the efficacy of the formulation because the arginine may form an insoluble arginine-calcium complex that has a poorer affinity for the tooth surface, and moreover that lowering the pH would reduce any effect the formulation might have on buffering cariogenic lactic acid in the mouth.

It would thus be beneficial to provide a dentifrice composition containing a cannabinoid and a basic amino acid.

### BRIEF SUMMARY

Provided herein are oral care compositions comprising both a cannabinoid (e.g., cannabidiol) and a basic amino acid (e.g., arginine). Without being bound by theory, it is believed that oral care compositions comprising a cannabinoid (e.g., CBD) and a basic amino acid (e.g., arginine) possess anti-inflammatory and antibacterial properties that can be used in the oral cavity. Thus, it is believed such a composition would provide an effective treatment against many oral diseases and disorders, such as gingivitis, abscesses, and cold sores. Moreover, the inventors have surprisingly demonstrated that cannabidiol in combination with a basic amino acid, e.g., arginine, can enhance the efficacy of arginine in suppressing bacterial cellular metabolism.

Thus, in a first aspect, the present disclosure provides an oral care composition comprising:
a) A basic amino acid (e.g., arginine or lysine);
b) a cannabinoid source (e.g., a cannabinoid source comprising cannabidiol); and
c) an orally acceptable vehicle.

In some embodiments, the amino acid is arginine in free or orally acceptable salt form. In some embodiments the cannabinoid is cannabidiol (CBD). Related methods of making and use are further provided.

### DETAILED DESCRIPTION

As used herein, the term “oral composition” means the total composition that is delivered to the oral surfaces. The composition is further defined as a product which, during the normal course of usage, is not, the purposes of systemic administration of particular therapeutic agents, intentionally swallowed but is rather retained in the oral cavity for a time sufficient to contact substantially all of the dental surfaces and/or oral tissues for the purposes of oral activity. Examples of such compositions include, but are not limited to, toothpaste or a dentifrice, a mouthwash or a mouth rinse, a topical oral gel, a denture cleanser, sprays, powders, strips, floss and the like.

As used herein, the term “dentifrice” means paste, or liquid formulations unless otherwise specified. The dentifrice composition can be in any desired form such as deep striped, surface striped, multi-layered, having the gel surrounding the paste, or any combination thereof. Alternatively, the oral composition may be dual phase dispensed from a separated compartment dispenser.

### COMPOSITIONS OF THE PRESENT DISCLOSURE

In one aspect the invention is an oral care composition (Composition 1) comprising:
a) an amino acid source;
b) a cannabinoid source; and
c) an orally acceptable vehicle.

For example, the invention contemplates any of the following compositions (unless otherwise indicated, values are given as percentage of the overall weight of the composition):

1.1 Composition 1, wherein the amino acid source comprises an amino acid selected from the group consisting of arginine, L-arginine, cysteine, leucine, isoleucine, lysine, L-lysine, alanine, asparagine, aspartate, phenylalanine, glutamate, glutamic acid, threonine, glutamine, tryptophan, glycine, valine, proline, serine, tyrosine, histidine, and mixtures thereof.
1.2 Any of the preceding compositions, wherein the amino acid has the configuration (e.g., L-arginine).
1.3 Any of the preceding compositions, wherein the amino acid source comprises a basic amino acid.
1.4 Any of the preceding compositions, wherein the amino acid source comprises an amino acid selected from the group consisting of arginine, lysine, glycine and combinations thereof.
1.5 Any of the preceding compositions, wherein the amino acid source comprises arginine.
1.6 Any of the preceding compositions wherein the amino acid source comprises an amino acid that is provided in the form of a di- or tri-peptide comprising arginine, or salts thereof.
1.7 Any of the preceding compositions wherein the amino acid source comprises arginine, and wherein the arginine is present in an amount corresponding to 0.1% to 10%, e.g., 0.1 wt. % to 5.0 wt. % of the total composition weight, about e.g., 0.5%, 1.0% 1.5%, 2.0%, 2.5%, or 3.0%, wherein the weight of the basic amino acid is calculated as free form.
1.8 Any of the preceding compositions wherein the amino acid source comprises arginine from 0.1 wt. % to 5.0 wt. %. (e.g., about 1.5 wt %) (e.g., about 3% by wt.).
1.9 Any of the preceding compositions wherein the amino acid source comprises arginine from about 1.5 wt. %.
1.10 Any of the preceding compositions wherein the amino acid source comprises L-arginine.
1.11 Any of the preceding compositions wherein the amino acid source comprises arginine in free form.
1.12 Any of the preceding compositions wherein the amino acid source comprises arginine in partially or wholly in salt form.
1.13 Any of the preceding compositions wherein the amino acid comprises an amino acid in the form of a salt selected from arginine phosphate, arginine hydrochloride or arginine bicarbonate.
1.14 Any of the preceding compositions wherein the amino acid source is arginine ionized by neutralization with an acid or a salt of an acid.
1.15 Any of the preceding compositions wherein the amino acid source comprises arginine phosphate.
1.16 Any of the preceding compositions wherein the amino acid source comprises arginine hydrochloride.
1.17 Any of the preceding compositions wherein the amino acid source comprises arginine bicarbonate.
1.18 Any of the preceding compositions, wherein the cannabinoid source comprises one or more cannabinoids selected from cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (THCV), Δ⁹-tetrahydrocannabinol (THC), tetrahydrocannabinolic acid (THCA) and combinations thereof.
1.19 Any of the preceding compositions, wherein the cannabinoid is a non-psychoactive cannabinoid.
1.20 Any of the preceding compositions, wherein the cannabinoid comprises less than 0.3 wt. % Δ⁹-tetrahydrocannabinol (THC) relative to the total weight of the composition.
1.21 Any of the preceding compositions, wherein the composition comprises less than 0.1 wt. % Δ⁹-tetrahydrocannabinol (THC) relative to the total weight of the composition.
1.22 Any of the preceding compositions, wherein the composition comprises less than 0.01 wt. % Δ⁹-tetrahydrocannabinol (THC) relative to the total weight of the composition.
1.23 Any of the preceding compositions, wherein the composition is substantially free of Δ⁹-tetrahydrocannabinol (THC).
1.24 Any of the preceding compositions, wherein the cannabinoid source comprises one or more cannabinoid selected from: cannabichromene (CBC), cannabigerol (CBG), cannabidiol (CBD), and cannabinol (CBN), and combinations thereof.
1.25 Any of the preceding compositions, wherein the cannabinoid source comprises cannabidiol (CBD).
1.26 Any of the preceding compositions, wherein the cannabinoid source is present in an amount of 0.001 wt. % to 1.0 wt. %, 0.01 wt. % to 0.8 wt. % (e.g., 0.05% by wt.), 0.1% to 0.5%, 0.2 wt. % to 0.4 wt. %, or about 0.1 wt. %, about 0.2 wt. %, about 0.3 wt. %, about 0.4 wt. %, or about 0.5 wt. %, relative to the total weight of the composition.
1.27 Any of the preceding compositions, wherein the cannabinoid source comprises:

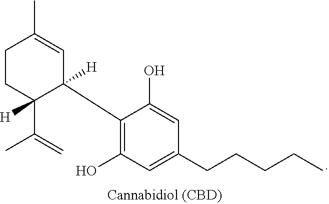 [SMILES: C=C(C)[C@@H]1CCC(C)=C[C@H]1c1c(O)cc(CCCCC)cc1O]

1.28 Any of the preceding compositions, wherein the cannabinoid source comprises hemp seed oil (HSO) or Cannabis sativa seed oil (CSO) (e.g., wherein the HSO or CSO is a carrier for one or more cannabinoids).
1.29 Any of the preceding compositions, further comprising an abrasive or particulate selected from alumina, aluminum hydroxide, calcium carbonate, precipitated calcium carbonate, dicalcium phosphate, mica, sodium bicarbonate, calcium pyrophosphate or combinations thereof.
1.30 The preceding composition, wherein the abrasive or particulate is a calcium abrasive.
1.31 The preceding composition, wherein the abrasive or particulate is a calcium abrasive selected from the group consisting of calcium carbonate, precipitated calcium carbonate, dicalcium phosphate calcium pyrophosphate or combinations thereof.
1.32 The preceding composition, wherein the abrasive is dicalcium phosphate.
1.33 The preceding composition, wherein the abrasive is dicalcium phosphate dihydrate.
1.34 Any of the preceding compositions, comprising an abrasive or particulate in an amount of about 10 to 90 wt. %, about 20 to 70 wt. %, about 30 to 50 wt. %, or about 35 to 45 wt. % (e.g., about 40%), calculated relative to the total weight of the composition.
1.35 The preceding composition, wherein the abrasive or particulate is present in an amount of about 30 to 50 wt. % (e.g., about 40%), calculated relative to the total weight of the composition.
1.36 The preceding composition, wherein the abrasive or particulate is present in an amount of about 35 to 45 wt. % (e.g., about 40%), calculated relative to the total weight of the composition.
1.37 The preceding composition, wherein the abrasive or particulate is present in an amount of about 40%, calculated relative to the total weight of the composition.
1.38 Any of the preceding compositions, comprising silica wherein the silica is used as a thickening agent, e.g., particle silica.
1.39 Any of the preceding compositions, wherein the orally acceptable vehicle comprises one or more of water, a thickener, a buffer, a humectant, a surfactant, a sweetener, a pigment, a dye, an anti-caries agent, an anti-bacterial, a whitening agent, a desensitizing agent, a vitamin, a preservative, an enzyme, and mixtures thereof.
1.40 Any of the preceding compositions, wherein the composition comprises a humectant selected from glycerin, sorbitol, xylitol, propylene glycol or combinations thereof.
1.41 Any of the preceding compositions, comprising a humectant in an amount of 15 to 70 wt. % or 30 to 65 wt. %, based on the total weight of the composition.
1.42 Any of the preceding compositions wherein the pH of the composition is between 6.5 and 7.5.
1.43 Any of the preceding compositions, wherein the pH of the composition is 6.5, 7.0 or 7.3.
1.44 Any of the preceding compositions, further comprising a fluoride source selected from: sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and combinations thereof.
1.45 Any of the preceding compositions, wherein the composition comprises a fluoride source present in an amount of 0.01 wt. % to 2 wt. % (e.g., 0.1 wt %-1.0 wt. %) of the total composition weight.
1.46 Any of the preceding compositions, wherein the composition comprises more than one fluoride source.
1.47 Any of the preceding compositions, wherein the composition comprises a combination of sodium fluoride and sodium monofluorophosphate.
1.48 Any of the preceding compositions, wherein the composition comprises a combination of sodium fluoride present in an amount of about 0.01 to 0.2 wt %, based on the total weight of the composition, and sodium monofluorophosphate present in an amount of about 0.5 to 1.0 wt. %, based on the total weight of the composition.
1.49 Any of the preceding compositions wherein the fluoride source provides fluoride ion in an amount of from 50 to 25,000 ppm (e.g., 750-7000 ppm, e.g., 1000-5500 ppm, e.g., about 500 ppm, 1000 ppm, 1100 ppm, 2800 ppm, 5000 ppm, or 25000 ppm).
1.50 Any of the preceding compositions, wherein the fluoride source comprises sodium fluoride.
1.51 Any of the preceding compositions further comprising an effective amount of one or more alkali phosphate salts, e.g., sodium, potassium or calcium salts, e.g., selected from alkali dibasic phosphate and alkali pyrophosphate salts, e.g., alkali phosphate salts selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, disodium hydrogenorthophosphate, monosodium phosphate, pentapotassium triphosphate and mixtures of any of two or more of these, e.g., in an amount of 0.01-20%, e.g., 0.1-8%, e.g., e.g., 0.1 to 5%, e.g., 0.3 to 2%, e.g., 0.3 to 1%, e.g about 0.01%, about 0.1%, about 0.5%, about 1%, about 2%, about 5%, about 6%, by weight of the composition.
1.52 Any of the preceding compositions comprising tetrapotassium pyrophosphate, disodium hydrogenorthophosphate, monosodium phosphate, and pentapotassium triphosphate.
1.53 Any of the preceding compositions comprising a polyphosphate.
1.54 The preceding composition, wherein the polyphosphate is tetrasodium pyrophosphate.
1.55 The preceding composition, wherein the tetrasodium pyrophosphate is from 0.01-1.0 wt % (e.g., about 0.25 wt %).
1.56 Any of the preceding compositions further comprising a nonionic surfactant, wherein the nonionic surfactant is in an amount of from 0.5-5%, selected from poloxamers (e.g., poloxamer 407), polysorbates (e.g., polysorbate 20), polyoxyl hydrogenated castor oil (e.g., polyoxyl 40 hydrogenated castor oil), and mixtures thereof.
1.57 The preceding composition, wherein the poloxamer nonionic surfactant has a polyoxypropylene molecular mass of from 3000 to 5000 g/mol and a polyoxyethylene content of from 60 to 80 mol %, e.g., the poloxamer nonionic surfactant comprises poloxamer 407.
1.58 Any of the preceding compositions further comprising sorbitol, wherein the sorbitol is in a total amount of 10-40% (e.g., about 23%).
1.59 Any of the preceding compositions, further comprising one or more zinc ion source(s) selected from zinc oxide, zinc citrate, zinc lactate, zinc phosphate and combinations thereof.
1.60 The preceding composition, wherein the one or more zinc ion source(s) comprises or consists of a combination of zinc oxide and zinc citrate.
1.61 The preceding composition, wherein the ratio of the amount of zinc oxide (e.g., wt. %) to zinc citrate (e.g., wt %) is from 1.5:1 to 4.5:1 (e.g., 2:1, 2.5:1, 3:1, 3.5:1, or 4:1).
1.62 Either of the two preceding compositions, wherein the zinc citrate is in an amount of from 0.25 to 1.0 wt % (e.g., 0.5 wt. %) and zinc oxide may be present in an amount of from 0.75 to 1.25 wt % (e.g., 1.0 wt. %) based on the weight of the oral care composition.
1.63 Any of the preceding compositions, wherein the zinc ion source comprises zinc citrate in an amount of about about 0.5 wt %.
1.64 Any of the preceding compositions, wherein the zinc ion source comprises zinc oxide in an amount of about 1.0 wt %.
1.65 Any of the preceding compositions, wherein the one or more zinc ion source(s) comprises zinc citrate in an amount of about about 0.5 wt % and zinc oxide in an amount of about 1.0 wt %.
1.66 Any of the preceding compositions, further comprising one or more stannous ion source(s).
1.67 The preceding composition, wherein the one or more stannous ion source(s) is selected from stannous fluoride, stannous chloride, stannous pyrophosphate, organic stannous carboxylate salts such as stannous formate, acetate, gluconate, lactate, tartrate, oxalate, malonate and citrate, stannous ethylene glyoxide, or a mixture thereof.
1.68 The preceding compositions, wherein the one or more stannous ion source is present in an amount of 0.1% by wt.-2.0% by wt.
1.69 Any of the preceding compositions, wherein the one or more stannous ion source comprises stannous fluoride.
1.70 Any of the preceding compositions further comprising an additional ingredient selected from: benzyl alcohol, Methylisothizolinone (“MIT”), Sodium bicarbonate, sodium methyl cocoyl tartrate (tauranol), lauryl alcohol, and polyphosphate.
1.71 Any of the preceding compositions, wherein the composition further comprises a copolymer.
1.72 The preceding composition, wherein the copolymer is a PVM/MA copolymer.
1.73 The preceding composition, wherein the PVM/MA copolymer comprises a 1:4 to 4:1 copolymer of maleic anhydride or acid with a further polymerizable ethylenically unsaturated monomer; for example, 1:4 to 4:1, e.g. about 1:1.
1.74 The preceding composition, wherein the further polymerizable ethylenically unsaturated monomer comprises methyl vinyl ether (methoxyethylene).
1.75 Any of compositions 1.50-1.52, wherein the PVM/MA copolymer comprises a copolymer of methyl vinyl ether/maleic anhydride, wherein the anhydride is hydrolyzed following copolymerization to provide the corresponding acid.
1.76 Any of compositions 1.50-1.53, wherein the PVM/MA copolymer comprises a GANTREZ® polymer (e.g., GANTREZ® S-97 polymer).
1.77 Any of the preceding compositions, wherein the composition comprises a thickening agent selected from the group consisting of carboxyvinyl polymers, carrageenan, xanthan, hydroxyethyl cellulose and water soluble salts of cellulose ethers (e.g., sodium carboxymethyl cellulose and sodium carboxymethyl hydroxyethyl cellulose).
1.78 Any of the preceding compositions further comprising sodium carboxymethyl cellulose (e.g., from 0.5 wt. %-1.5 wt. %).
1.79 Any of the preceding compositions comprising from 5%-40%, e.g., 10%-35%, e.g., about 15%, 25%, 30%, and 35% water.
1.80 Any of the preceding compositions comprising an additional antibacterial agent selected from halogenated diphenyl ether (e.g. triclosan), herbal extracts and essential oils (e.g., rosemary extract, tea extract, magnolia extract, thymol, menthol, eucalyptol, geraniol, carvacrol, citral, honokiol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC), phenolic antiseptics, hexetidine, octenidine, sanguinarine, povidone iodine, delmopinol, salifluor, metal ions (e.g., copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and mixtures of any of the foregoing.
1.81 Any of the preceding compositions comprising an antioxidant, e.g., selected from the group consisting of Co-enzyme Q10, PQQ, Vitamin C, Vitamin E, Vitamin A, BHT, anethole-dithiothione, and mixtures thereof.
1.82 Any of the preceding compositions comprising a whitening agent.
1.83 Any of the preceding compositions comprising a whitening agent selected from a whitening active selected from the group consisting of peroxides, metal chlorites, perborates, percarbonates, peroxyacids, hypochlorites, and combinations thereof.
1.84 Any of the preceding compositions further comprising hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example, calcium peroxyphosphate, sodium perborate, sodium carbonate peroxide, sodium peroxyphosphate, and potassium persulfate), or hydrogen peroxide polymer complexes such as hydrogen peroxide-polyvinyl pyrrolidone polymer complexes.
1.85 Any of the preceding compositions further comprising an agent that interferes with or prevents bacterial attachment, e.g. ethyl lauroyl arginiate (ELA) or chitosan.
1.86 Any of the preceding oral compositions, wherein the oral composition may be any of the following oral compositions selected from the group consisting of: a toothpaste or a dentifrice, a mouthwash or a mouth rinse, a topical oral gel (e.g., an oral gel meant for office or professional use), a chewing gum, a dental tray application, mouth spray, foam, tablet, powder, a non-abrasive gel, a mousse, a denture cleanser, a coated or impregnated immediate or delayed release oral adhesive strip or patch, and a coated or impregnated oral wipe or swab.
1.87 A composition obtained or obtainable by combining the ingredients as set forth in any of the preceding compositions.
1.88 Any of the preceding oral care compositions, wherein the cannabinoid source comprises substantially pure cannabidiol (CBD) (e.g., wherein the amount of CBD (by wt %) is 90%, 95%, or 99% or more (by wt %) of the total amount of cannabinoids in the oral care composition, by total wt % of the oral care composition).
1.89 Any of the preceding compositions, wherein the cannabinoid source comprises a cannabinoid selected from cannabichromene (CBC), cannabigerol (CBG), cannabidiol (CBD), and cannabinol (CBN), and combinations thereof.
1.90 Any of the preceding compositions, wherein the cannabinoid source comprises:

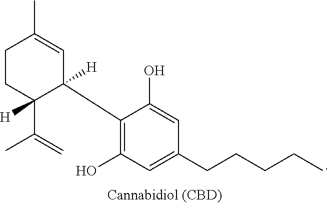 [SMILES: C=C(C)[C@@H]1CCC(C)=C[C@H]1c1c(O)cc(CCCCC)cc1O]

1.91 Any of the preceding compositions, wherein the cannabinoid source comprises hemp seed oil (HSO) or Cannabis sativa seed oil (CSO) or hemp oil, and wherein the HSO or CSO or hemp oil is a carrier for one or more cannabinoids.
1.92 The composition of 1.91, wherein the one or more cannabinoid is selected from cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (THCV), Δ⁹-tetrahydrocannabinol (THC), tetrahydrocannabinolic acid (THCA), and combinations thereof.
1.93 The composition of 1.92 wherein the cannabinoid source comprises cannabidiol (CBD).
1.94 Any of the preceding compositions comprising:
a. arginine (e.g., about 1.5%)
b. a cannabinoid source comprising cannabidiol (e.g., about 0.5% by wt of a cannabinoid source comprising Cannabis sativa seed oil (CSO), wherein the CSO further comprises about 5% by wt of cannabidiol (wherein the wt. % of CBD is based on the total weight wt. of the CSO))
   1.95 Any of Composition 1-1.93, wherein the composition comprises:
a. arginine (e.g., from about 1% by wt. to 5% by wt.) (e.g. about 1.5%) (e.g. about 3% by wt.)
b. zinc citrate (e.g., zinc citrate trihydrate) (e.g., about 0.5% zinc citrate) and zinc oxide (e.g., about 1.0% by wt.); and
c. a cannabinoid source comprising cannabidiol (e.g., about 0.5% by wt of a cannabinoid source comprising Cannabis sativa seed oil (CSO), wherein the CSO further comprises about 5% by wt of cannabidiol (wherein the wt. % of CBD is based on the total weight wt. of the CSO));
d. sodium fluoride
   1.96 Any of Composition 1-1.93, wherein the composition comprises
a. arginine (e.g., about 1.5%)
b. zinc citrate (e.g., zinc citrate trihydrate) (e.g., about 0.5% zinc citrate) and zinc oxide (e.g., about 1.0% by wt.);
c. stannous fluoride; and
d. a cannabinoid source comprising cannabidiol (e.g., about 0.5% by wt of a cannabinoid source comprising Cannabis sativa seed oil (CSO), wherein the CSO further comprises about 5% by wt of cannabidiol (wherein the wt. % of CBD is based on the total weight wt. of the CSO))
   1.97 Any composition of 1-1.93, wherein the composition comprises:
a. arginine (e.g., about 1.5% by wt.)
b. zinc phosphate (about 1.0% by wt.)
c. stannous fluoride; and
d. a cannabinoid source comprising cannabidiol (e.g., about 0.5% by wt of a cannabinoid source comprising Cannabis sativa seed oil (CSO), wherein the CSO further comprises about 5% by wt of cannabidiol (wherein the wt. % of CBD is based on the total weight wt. of the CSO))
   1.98 Any of the preceding oral care compositions, wherein the cannabinoid source comprises one or more cannabinoids selected from: cannabichromene (CBC), cannabigerol (CBG), cannabidiol (CBD), and/or cannabinol (CBN), and wherein the one or more cannabinoids are present in an amount of 0.001 wt. % to 3.0 wt. % (e.g., 0.0025%), 0.02 wt. % to 0.8 wt. %, 0.1% to 0.5%, 0.2 wt. % to 0.4 wt. %, 0.005 wt. %, 0.01 wt. %, 0.025 wt. %, 0.05 wt. %, or 0.3 wt. % relative to the total weight of the composition.
   1.99 Any of the preceding compositions, wherein the cannabinoid source comprises cannabidiol (CBD).
   1.100 Any of the preceding compositions, comprising cannabidiol in an amount of 0.005 wt. % to 3.0 wt. %, 0.01 wt. % to 0.8 wt. %, 0.1% to 0.5%, 0.2 wt. % to 0.4 wt. %, about 0.005 wt. %, about 0.01 wt. %, about 0.025 wt. %, about 0.05 wt. %, about 0.1 wt. %, about 0.2 wt. %, or about 0.3 wt. % relative to the total weight of the composition.
   1.101 Any of the preceding oral compositions, wherein the oral composition may be any of the following oral compositions selected from the group consisting of: a toothpaste or a dentifrice, a mouthwash or a mouth rinse, a topical oral gel (e.g., an oral gel meant for office or professional use), a chewing gum, a dental tray application, mouth spray, foam, tablet, powder, a non-abrasive gel, a mousse, a denture cleanser, a coated or impregnated immediate or delayed release oral adhesive strip or patch, and a coated or impregnated oral wipe or swab.

In various embodiments, the present disclosure also provides for a composition obtained or obtainable by combining the ingredients as set forth in any of the preceding compositions.

Additionally, the present disclosure provides for a composition for use as set forth in any of the preceding compositions.

The invention further comprises the use of an amino acid and a cannabinoid in the manufacture of a Composition of the Invention, e.g., for use in any of the indications set forth in the above method of Composition 1, et seq.

In a further aspect, the invention contemplates a method of decreasing mitochondrial respiration (e.g., oxygen consumption rate) and/or glycolysis (e.g., measured by extracellular acidification rate) in an oral biofilm of a subject in need thereof, wherein the method comprises administering any of Composition 1.0 et seq to the oral cavity of the subject. In one aspect, the invention contemplates a method of decreasing mitochondrial respiration (e.g., oxygen consumption rate) and/or glycolysis (e.g., measured by extracellular acidification rate) in an oral biofilm of a subject in need thereof, wherein the method comprises administering any of Composition 1.0 et seq to the oral cavity of the subject; and wherein the biofilm comprises S. mutans. In one aspect, the subject in need thereof has an elevated concentration or amount of S. mutans bacteria relative to a reference standard.

The term “cannabinoid” as used herein may refer to any compound that interacts with a cannabinoid receptor and other cannabinoid mimetics, including, but not limited to, certain tetrahydropyran analogs (Δ⁹-tetrahydrocannabinol, Δ⁸-tetrahydrocannabinol, 6,6,9-trimythel-3-pentyl-6H-dibenzo[b,d]pyran-1-ol, 3-(1,1-dimethylheptyl)-6,6a7,8,10,10a-hexahydro-1-1hydroxy-6,6-dimythel-9H-dibezo[b,d]pyran-9-ol, (−)-(3S,4S)-7-hydroxy-delta-6-tetrahydrocannabinol-1,1-dimethylheptyl, (+)-(3S,4S)-7-hydroxy-Δ-6-tetrahydrocannabinol, and Δ⁸-tetrahydrocannabinol-11-oic acid); certain piperidine analogs (e.g., (−)-(6S,6aR,9R,10aR)-5,6,6a,7,8,9,10,10a-octahydro-6-methyl-1-3-[(R)-1-methyl-4-phenylbutoxy]-1,9-phenanthridinediol 1-acetate)); certain aminoalkylindole analogs (e.g., (R)-(+)-[2,3-dihydro-5-methyl-3-(4-morpholinylm-ethyl)-pyrrolo[1,2,3,-de]-1,4-benzoxazin-6-yl]-1-naphthelenyl-methanone); certain open pyran-ring analogs (e.g., 2-[3-methyl-6-(1-methylethenyl-2-cyclohexen-1-yl]-5-pentyl-1,3-benzendi-ol, and 4-(1,1-dimethylheptyl)-2,3′-dihydroxy-6′-α-(3-hydroxypropyl)-1′,-2′,3′,4′,5′,6′-hexahydrobiphenyl), their salts, solvates, metabolites, and metabolic precursors. In one aspect, the cannabinoid source (e.g., any of Composition 1.0 et seq) of comprises cannabidiol with the following structure:

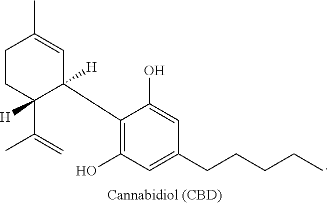 [SMILES: C=C(C)[C@@H]1CCC(C)=C[C@H]1c1c(O)cc(CCCCC)cc1O]

The term “hemp seed oil” or “Cannabis sativa seed oil” refer to oil derived from hemp seed or Cannabis sativa seed. The term “hemp oil” refers to oil derived from Cannabis sativa (or hemp) flower, leaf, stem, or the whole plant, wherein the Cannabis sativa or hemp plant contains less than 0.3% by wt. THC. The term “hemp oil” refers to oil derived from Cannabis sativa (or hemp) flower, leaf, stem, or the whole plant, wherein the Cannabis sativa or hemp plant contains less than 0.3% by wt. THC.

The term “cannabidiol” as used herein refers to cannabidiol and cannabidiol derivatives. As used in this application, cannabidiol may be obtained from industrial hemp extract with a trace amount of THC (e.g., less than 0.3% by weight) or from Cannabis extract using high-CBD Cannabis cultivars.

Cannabinoids utilized in the present invention may be in liquid form, as a natural (or additional) constituent of hemp oil, hemp seed oil or Cannabis sativa seed oil. “Hemp seed oil” (HSO) or “Cannabis sativa seed oil” (CSO) are used herein interchangeably. Hemp oil, HSO, or CSO, are harvested by cold pressing the seeds and the plants of the Cannabis sativa species. The resulting oil is extracted using CO₂ extraction or solvent extraction process, and may be further concentrated by distillation. Choice of cultivars may give different cannabinoid concentrations, but preferably, the targeted cannabinoids are cannabidiol (CBD) and cannabigerol (CBG). Other cannabinoids such as THC and cannabichromene (CBC) may also be present in hemp oil or Cannabis oil. Further isolation of these cannabinoids may result in solid, purified cannabinoids.

In certain embodiments, cannabinoids of the present invention may be present as isolates or extracts from the plants of the Cannabis sativa species.

In a preferred embodiment, toothpaste is manufactured with one or more cannabinoids incorporated for anti-bacterial effects. In this embodiment, the one or more cannabinoids are naturally derived or artificially derived.

in one aspect, Hemp oil, hemp seed oil, or Cannabis sativa seed oil can serve as delivery vehicle for the cannabinoid source. When cannabinoids are provided in hemp oil or hemp seed oil or Cannabis sativa seed oil, the hemp oil, hemp seed oil, or Cannabis sativa seed oil may contain up to 85% impurities, including fatty acids and other plant impurities. The extracted oil is then distilled to increase the cannabinoid concentration. Impurities in hemp oil, hemp seed oil and Cannabis sativa seed oil may be fatty acids such as linoleic acid and α-linoleic acid, which are natural components of hemp oil or Cannabis oil, β-caryophyllene, myrcene, and β-sitosterol.

In this embodiment, cannabinoids provided as hemp oil, hemp seed oil or Cannabis sativa seed oil may contain impurities in an amount of less than 85%, less than 80%, less than 75%, less than 70%, less than 65%, less than 55%, less than 50%, less than 45%, less than 40%, or less than 35% by weight.

The oral care compositions may further include one or more fluoride ion sources, e.g., soluble fluoride salts. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al., each of which are incorporated herein by reference. Representative fluoride ion sources used with the present invention (e.g., Composition 1.0 et seq.) include, but are not limited to, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes sodium fluoride, sodium monofluorophosphate as well as mixtures thereof. Where the formulation comprises calcium salts, the fluoride salts are preferably salts wherein the fluoride is covalently bound to another atom, e.g., as in sodium monofluorophosphate, rather than merely ionically bound, e.g., as in sodium fluoride.

The invention may in some embodiments contain anionic surfactants, e.g., the Compositions of Composition 1.0, et seq., for example, water-soluble salts of higher fatty acid monoglyceride monosulfates, such as the sodium salt of the monosulfated monoglyceride of hydrogenated coconut oil fatty acids such as sodium N-methyl N-cocoyl taurate, sodium cocomo-glyceride sulfate; higher alkyl sulfates, such as sodium lauryl sulfate; higher alkyl-ether sulfates, e.g., of formula CH₃(CH₂)ₘCH₂(OCH₂CH₂)ₙOS0₃X, wherein m is 6-16, e.g., 10, n is 1-6, e.g., 2, 3 or 4, and X is Na or, for example sodium laureth-2 sulfate (CH₃(CH2)₁₀CH₂(OCH₂CH₂)₂OS0₃Na); higher alkyl aryl sulfonates such as sodium dodecyl benzene sulfonate (sodium lauryl benzene sulfonate); higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate. By “higher alkyl” is meant, e.g., C₆-₃o alkyl. In particular embodiments, the anionic surfactant (where present) is selected from sodium lauryl sulfate and sodium ether lauryl sulfate. When present, the anionic surfactant is present in an amount which is effective, e.g., >0.001% by weight of the formulation, but not at a concentration which would be irritating to the oral tissue, e.g., 1%, and optimal concentrations depend on the particular formulation and the particular surfactant. In one embodiment, the anionic surfactant is present at from 0.03% to 5% by weight, e.g., 1.5%.

In another embodiment, cationic surfactants useful in the present invention can be broadly defined as derivatives of aliphatic quaternary ammonium compounds having one long alkyl chain containing 8 to 18 carbon atoms such as lauryl trimethylammonium chloride, cetyl pyridinium chloride, cetyl trimethylammonium bromide, di-isobutylphenoxyethyldimethylbenzylammonium chloride, coconut alkyltrimethylammonium nitrite, cetyl pyridinium fluoride, and mixtures thereof. Illustrative cationic surfactants are the quaternary ammonium fluorides described in U.S. Pat. No. 3,535,421, to Briner et al., herein incorporated by reference. Certain cationic surfactants can also act as germicides in the compositions.

Illustrative nonionic surfactants of Composition 1.0, et seq., that can be used in the compositions of the invention can be broadly defined as compounds produced by the condensation of alkylene oxide groups (hydrophilic in nature) with an organic hydrophobic compound which may be aliphatic or alkylaromatic in nature. Examples of suitable nonionic surfactants include, but are not limited to, the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials. In a particular embodiment, the composition of the invention comprises a nonionic surfactant selected from polaxamers (e.g., polaxamer 407), polysorbates (e.g., polysorbate 20), polyoxyl hydrogenated castor oils (e.g., polyoxyl 40 hydrogenated castor oil), betaines (such as cocamidopropylbetaine), and mixtures thereof.

Illustrative amphoteric surfactants of Composition 1.0, et seq., that can be used in the compositions of the invention include betaines (such as cocamidopropylbetaine), derivatives of aliphatic secondary and tertiary amines in which the aliphatic radical can be a straight or branched chain and wherein one of the aliphatic substituents contains about 8-18 carbon atoms and one contains an anionic water-solubilizing group (such as carboxyl ate, sulfonate, sulfate, phosphate or phosphonate), and mixtures of such materials.

The surfactant or mixtures of compatible surfactants can be present in the compositions of the present invention in 0.1% to 5%, in another embodiment 0.3% to 3% and in another embodiment 0.5% to 2% by weight of the total composition.

The oral care compositions of the invention may also include a flavoring agent. Flavoring agents which are used in the practice of the present invention include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint.

The flavoring agent is incorporated in the oral composition at a concentration of 0.01 to 1% by weight.

The oral care compositions of the invention also may include one or more chelating agents able to complex calcium found in the cell walls of the bacteria. Binding of this calcium weakens the bacterial cell wall and augments bacterial lysis.

Another group of agents suitable for use as chelating or anti-calculus agents in the present invention are the soluble pyrophosphates. The pyrophosphate salts used in the present compositions can be any of the alkali metal pyrophosphate salts. In certain embodiments, salts include tetra alkali metal pyrophosphate, dialkali metal diacid pyrophosphate, trialkali metal monoacid pyrophosphate and mixtures thereof, wherein the alkali metals are sodium or potassium. The salts are useful in both their hydrated and unhydrated forms. An effective amount of pyrophosphate salt useful in the present composition is generally enough to provide least 0.1 wt. % pyrophosphate ions, e.g., 0.1 to 3 wt 5, e.g., 0.1 to 2 wt %, e.g., 0.1 to 1 wt %, e.g., 0.2 to 0.5 wt %. The pyrophosphates also contribute to preservation of the compositions by lowering water activity.

The oral care compositions of the invention also optionally include one or more polymers, such as polyethylene glycols, polyvinyl methyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water soluble alkali metal (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene) having a molecular weight (M.W.) of about 30,000 to about 1,000,000. These copolymers are available for example as Gantrez AN 139 (M.W. 500,000), AN 1 19 (M.W. 250,000) and S-97 Pharmaceutical Grade (M.W. 70,000), of GAF Chemicals Corporation.

Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1 103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility.

A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000, described in U.S. Pat. No. 4,842,847, Jun. 27, 1989 to Zahid, incorporated herein by reference.

Another useful class of polymeric agents includes polyamine acids, particularly those containing proportions of anionic surface-active amino acids such as aspartic acid, glutamic acid and phosphoserine, as disclosed in U.S. Pat. No. 4,866,161 Sikes et al., incorporated herein by reference.

In preparing oral care compositions, it is sometimes necessary to add some thickening material to provide a desirable consistency or to stabilize or enhance the performance of the formulation. In certain embodiments, the thickening agents are carboxyvinyl polymers, carrageenan, xanthan gum, hydroxyethyl cellulose and water soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate or finely divided silica can be used as component of the thickening composition to further improve the composition's texture. In certain embodiments, thickening agents in an amount of about 0.5% to about 5.0% by weight of the total composition are used.

Natural calcium carbonate is found in rocks such as chalk, limestone, marble and travertine. It is also the principle component of egg shells and the shells of mollusks. The natural calcium carbonate abrasive of the invention is typically a finely ground limestone which may optionally be refined or partially refined to remove impurities. For use in the present invention, the material has an average particle size of less than 10 microns, e.g., 3-7 microns, e.g. about 5.5 microns. For example, a small particle silica may have an average particle size (D50) of 2.5-4.5 microns. Because natural calcium carbonate may contain a high proportion of relatively large particles of not carefully controlled, which may unacceptably increase the abrasivity, preferably no more than 0.01%, preferably no more than 0.004% by weight of particles would not pass through a 325 mesh, The material has strong crystal structure, and is thus much harder and more abrasive than precipitated calcium carbonate. The tap density for the natural calcium carbonate is for example between 1 and 1.5 g/cc, e.g., about 1.2 for example about 1.19 g/cc. There are different polymorphs of natural calcium carbonate, e.g., calcite, aragonite and vaterite, calcite being preferred for purposes of this invention. An example of a commercially available product suitable for use in the present invention includes Vicron® 25-11 FG from GMZ.

Precipitated calcium carbonate is generally made by calcining limestone, to make calcium oxide (lime), which can then be converted back to calcium carbonate by reaction with carbon dioxide in water. Precipitated calcium carbonate has a different crystal structure from natural calcium carbonate. It is generally more friable and more porous, thus having lower abrasivity and higher water absorption. For use in the present invention, the particles are small, e.g., having an average particle size of 1-5 microns, and e.g., no more than 0.1%, preferably no more than 0.05% by weight of particles which would not pass through a 325 mesh. The particles may for example have a D50 of 3-6 microns, for example 3.8=4.9, e.g., about 4.3; a D50 of 1-4 microns, e.g. 2.2-2.6 microns, e.g., about 2.4 microns, and a D10 of 1-2 microns, e.g., 1.2-1.4, e.g. about 1.3 microns. The particles have relatively high water absorption, e.g., at least 25 g/l00g, e.g. 30-70 g/l00g. Examples of commercially available products suitable for use in the present invention include, for example, Carbolag® 15 Plus from Lagos Industria Quimica.

In certain embodiments the invention may comprise additional calcium-containing abrasives, for example calcium phosphate abrasive, e.g., tricalcium phosphate (Ca₃(P0₄)₂), hydroxyapatite (Ca₁₀(P0₄)₆(OH)₂), or dicalcium phosphate dihydrate (CaHP0₄·2H₂0, also sometimes referred to herein as DiCal) or calcium pyrophosphate, and/or silica abrasives, sodium metaphosphate, potassium metaphosphate, aluminum silicate, calcined alumina, bentonite or other siliceous materials, or combinations thereof. Any silica suitable for oral care compositions may be used, such as precipitated silicas or silica gels. For example synthetic amorphous silica. Silica may also be available as a thickening agent, e.g., particle silica. For example, the silica can also be small particle silica (e.g., Sorbosil AC43 from PQ Corporation, Warrington, United Kingdom). However the additional abrasives are preferably not present in a type or amount so as to increase the RDA of the dentifrice to levels which could damage sensitive teeth, e.g., greater than 130.

Water is present in the oral compositions of the invention. Water, employed in the preparation of commercial oral compositions should be deionized and free of organic impurities. Water commonly makes up the balance of the compositions and includes 5% to 45%, e.g., 10% to 20%, e.g., 25-35%, by weight of the oral compositions. This amount of water includes the free water which is added plus that amount which is introduced with other materials such as with sorbitol or silica or any components of the invention. The Karl Fischer method is a one measure of calculating free water.

Within certain embodiments of the oral compositions, it is also desirable to incorporate a humectant to reduce evaporation and also contribute towards preservation by lowering water activity. Certain humectants can also impart desirable sweetness or flavor to the compositions. The humectant, on a pure humectant basis, generally includes 15% to 70% in one embodiment or 30% to 65% in another embodiment by weight of the composition.

Suitable humectants include edible polyhydric alcohols such as glycerine, sorbitol, xylitol, propylene glycol as well as other polyols and mixtures of these humectants. Mixtures of glycerine and sorbitol may be used in certain embodiments as the humectant component of the compositions herein.

In some embodiments, the compositions of the present disclosure contain a buffering agent. Examples of buffering agents include anhydrous carbonates such as sodium carbonate, sesquicarbonates, bicarbonates such as sodium bicarbonate, silicates, bisulfates, phosphates (e.g., monopotassium phosphate, dipotassium phosphate, tribasic sodium phosphate, sodium tripolyphosphate, phosphoric acid), citrates (e.g. citric acid, trisodium citrate dehydrate), pyrophosphates (sodium and potassium salts) and combinations thereof. The amount of buffeting agent is sufficient to provide a pH of about 5 to about 9, preferable about 6 to about 8, and more preferable about 7, when the composition is dissolved in water, a mouthrinse base, or a toothpaste base. Typical amounts of buffering agent are about 5% to about 35%, in one embodiment about 10% to about 30%, in another embodiment about 15% to about 25%, by weight of the total composition.

The amino acids of the present invention, in one aspect, can be basic amino acids. The basic amino acids which can be used in the compositions and methods of the invention (e.g., any of Composition 1.0 et seq) include not only naturally occurring basic amino acids, such as arginine, lysine, and histidine, but also any basic amino acids having a carboxyl group and an amino group in the molecule, which are water-soluble and provide an aqueous solution with a pH of 7 or greater.

Accordingly, basic amino acids include, but are not limited to, arginine, lysine, citrullene, ornithine, creatine, histidine, diaminobutanoic acid, diaminoproprionic acid, salts thereof or combinations thereof. In a particular embodiment, the basic amino acids are selected from arginine, citrullene, and ornithine.

In certain embodiments, the basic amino acid is arginine, for example, L-arginine, or a salt thereof.

The compositions of the invention are intended for topical use in the mouth and so salts for use in the present invention should be safe for such use, in the amounts and concentrations provided. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion.

The present invention in its method aspect involves applying to the oral cavity a safe and effective amount of the compositions described herein.

The compositions and methods according to the invention (e.g., Composition 1.0 et seq) can be incorporated into oral compositions for the care of the mouth and teeth such as toothpastes, transparent pastes, gels, mouth rinses, sprays and chewing gum.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls. It is understood that when formulations are described, they may be described in terms of their ingredients, as is common in the art, notwithstanding that these ingredients may react with one another in the actual formulation as it is made, stored and used, and such products are intended to be covered by the formulations described.

The following examples further describe and demonstrate illustrative embodiments within the scope of the present invention. The examples are given solely for illustration and are not to be construed as limitations of this invention as many variations are possible without departing from the spirit and scope thereof. Various modifications of the invention in addition to those shown and described herein should be apparent to those skilled in the art and are intended to fall within the appended claims.

### EXAMPLES

### Example 1

Samples with varying amounts of cannabidiol (CBD) are assessed for their anti-oxidation ability. The samples include hemp seed oil (HSO) with varying concentrations of CBD, where the amount of CBD in the HSO of each sample is 5% by wt. relative to the weight of the HSO. The HSO can be considered a delivery vehicle for the CBD.

Samples containing 0.05% HSO and CBD (5% CBD by wt. of HSO), 0.1% HSO and CBD (5% CBD by wt. of HSO), 0.2% HSO and CBD (5% CBD by wt. of HSO), and 0.5% HSO and CBD (5% CBD by wt. of HSO) are tested in an assay to assess anti-oxidation performance. The amount (%) of CBD is relative to weight of the HSO. The samples are compared to untreated samples (negative control) as well as samples with vitamin E raw material (positive control).

Samples with 0.5% HSO and CBD (5% CBD by wt. of HSO) show comparable anti-oxidation capability compared to samples that contain vitamin E. Untreated samples are not believed to demonstrate any anti-oxidative efficacy, while samples with 0.05% HSO and CBD (5% CBD by wt. of HSO), 0.1% HSO and CBD (5% CBD by wt. of HSO), and 0.2% HSO and CBD (5% CBD by wt. of HSO) demonstrate increasing anti-oxidation efficacy, respectively, compared to the untreated samples.

Total Antioxidant Capacity Assay Kit (Abeam Catalog #: ab65329) is used to test raw material (full spectrum Hemp seed oil with 5% CBD) to assess anti-oxidation capacity of raw material. In this assay Cu2+ is used as proxy for the ROS (Reactive Oxygen Species)/Oxidized form. The transfer of an electron from an antioxidant molecule converts Cu2+ (oxidized form) to Cu+1 (reduced form). Reduced Cu+ ion chelates with a colorimetric probe, giving a broad absorbance peak at 570 nm, which is proportional to the total antioxidant capacity. The kit gives antioxidant capacity in Trolox equivalents. Trolox, a water-soluble vitamin E analog, serves as an antioxidant standard.

Assay is conducted by using Cu2+ working solution (made by diluting 1 part of the Cu²⁺ reagent in 49 parts Assay Buffer). Place 100 μl of each sample and standard in a 96-well clear flat bottom plate. Add 100 μl of Cu²⁺ working solution to each well with samples or standards. After recommended incubation time plate is measured for absorbance at 570 nm. Data analysis is performed by creating a linear standard curve by plotting the concentration and absorbance of the standards. Standard curve is used to determine the concentration of the samples.

### Example 2

The assay described in Example 1 is conducted with toothpaste samples. The assay assesses a placebo toothpaste (i.e., which does not contain CBD), a CBD toothpaste, and vitamin E raw material. Similar to the results in Example 1, the CBD toothpaste will demonstrate comparable anti-oxidation capability compared to the vitamin E raw material (positive control).

### Example 3

Samples with varying amounts of cannabidiol (CBD) are assessed for their effects on cellular metabolism with and without various source of zinc. Using the Seahorse XF^{e} Extracellular Flux Analyzer, biofilm samples are grown for 48 hrs. The results are demonstrated in Tables 1, 2, and 3 below. The biofilms are harvested for culture. Diluted biofilm and toothpaste slurry are loaded to each well. Glycolysis—Extracellular Acidification Rate (ECAR) and Mitochondrial Respiration—Oxygen Consumption Rate (OCR) is measured for 25-50 cycles (˜360 minutes). The biofilm samples used to generate the results in Tables 1-3 were mixed species biofilm. The biofilm samples used to generate the results in Tables 4-5 were S. mutans specific biofilms. The “CBD Oil” is 5% CBD (by wt. of the CBD Oil) in Hemp seed oil.

Table 1 below demonstrates the effect of CBD and zinc combinations on bacterial metabolism (Oxygen Consumption Rate) in a mixed biofilm assay. Where the “OCR” is oxygen consumption rate units as measured using the Seahorse XF^{e} Extracellular Flux Analyzer.:

TABLE 1

| | Average |
|---|---|
| Sample | (OCR) (p mole/min) |
| Untreated (negative control) | 25,669.50 |
| 1.5% Zinc Lactate | 21,900.63 |
| 0.1% CBD oil* | 25,521.08 |
| 1.5% by wt. Zinc Lactate + 0.1% | 16,634.57 |
| CBD oil* | |
| 1.5% by wt. Zinc Lactate + 0.25% | 7,357.44 |
| CBD oil* | |
| 1.5% by wt. Zinc Lactate + 0.5% | 7,525.59 |
| CBD oil* | |

*Where the CBD oil contains 5% CBD by wt. of the CBD oil.

As demonstrated in Table 1, slurries with 1.5% zinc lactate and 0.1%, 0.25%, or 0.5% CBD oil (*containing 5% CBD by wt. of the oil) unexpectedly decrease the OCR of the mixed species biofilm samples when compared to samples that only contain zinc lactate or only contain 0.1% CBD oil. Note that the samples listed in Table 1 only include those ingredients listed in the table.

In a separate experiment, Table 2 demonstrates that CBD enhances the effect of zinc citrate and zinc oxide slurry combinations on the acid production rate in mixed species biofilms:

TABLE 2

| Sample | ECAR values mpH/min |
|---|---|
| Untreated (Negative Control) | 3,800.0 |
| 0.25% CBD oil* | 3,191.9 |
| 0.5% by wt. Zinc Citrate + | 3000 |
| 1% by wt. Zinc Oxide | |
| 0.25% CBD oil* + 0.5% ZnC + | 1968.0 |
| 1% ZnO | |

*Where the CBD oil contains 5% CBD by wt. of the CBD oil.

As demonstrated in Table 2, slurries with 0.5% zinc citrate, 1% zinc oxide and 0.25% CBD Oil (5% CBD by wt. of the oil) unexpectedly lower the ECR of the mixed species biofilm samples when compared to samples that only contain only zinc citrate and zinc oxide (0.5% by wt and 1% by wt., respectively) or only contain 0.25% CBD oil (5% CBD by wt. of the oil). Note that the samples listed in Table 2 only include those ingredients listed in the table.

In a separate experiment, Table 3 demonstrates that CBD enhances the effect of stannous slurry combinations on the suppression of bacterial metabolic rate (oxygen consumption rate) in mixed species biofilms:

TABLE 3

| | Average |
|---|---|
| Sample | (OCR) (p mole/min) |
| Untreated (negative control) | 11489.9 |
| 0.2% by wt. Stannous Fluoride | 10440.7 |
| 0.15% by wt. CBD oil* | 8659.6 |
| 0.2% by wt. Stannous Fluoride + 0.15% CBD oil* | 1626.9 |

*Where the CBD oil contains 5% CBD by wt. of the CBD oil.

As demonstrated in Table 3, slurries with 0.2% stannous fluoride and 0.15% CBD Oil (5% CBD by wt. of the oil) unexpectedly lower the OCR of the mixed species biofilm samples when compared to samples that only contain only stannous fluoride or only contain 0.15% CBD oil (5% CBD by wt. of the oil). Note that the samples listed in Table 3 only include those ingredients listed in the table.

In a separate experiment, Table 4 demonstrates that CBD enhances the effect of arginine slurry combinations on the suppression of bacterial metabolic rate (oxygen consumption rate) specifically in S. mutans biofilms:

TABLE 4

| Sample Type | OCR Value (p mole/min) |
|---|---|
| Untreated | 1449.50 |
| 0.05% by wt. CBD oil* | 827.42 |
| 1% Arg | 518.58 |
| 0.05% CBD oil* + 1% Arg | 90.4 |

*Where the CBD oil contains 5% CBD by wt. of the CBD oil.

As demonstrated in Table 4, slurries with 0.2% stannous fluoride and 0.15% CBD Oil (5% CBD by wt. of the oil) unexpectedly lower the OCR of the S. mutans biofilm samples when compared to samples that only contain only stannous fluoride or only contain 0.15% CBD oil (5% CBD by wt. of the oil). Note that the samples listed in Table 4 only include those ingredients listed in the table. The biofilm samples used to generate the results in Tables 4 were S. mutans specific biofilms.

In a separate experiment, Table 5 demonstrates that CBD enhances the effect of arginine slurry combinations on the suppression of extracellular acidification rate (ECAR) specifically in S. mutans biofilms:

TABLE 5

| Sample Type | ECR Value (mpH/min) |
|---|---|
| Untreated (negative control) | 701.2 |
| 0.05% by wt. CBD oil* | 645.2 |
| 1% by wt. Arg | 518.6 |
| 0.05% CBD oil* + 3% Arg | 90.4 |

*Where the CBD oil contains 5% CBD by wt. of the CBD oil.

As demonstrated in Table 5, slurries with 0.2% stannous fluoride and 0.15% CBD Oil (5% CBD by wt. of the oil) unexpectedly lower the OCR of the S. mutans biofilm samples when compared to samples that only contain only stannous fluoride or only contain 0.15% CBD oil (5% CBD by wt. of the oil). Note that the samples listed in Table 5 only include those ingredients listed in the table. The biofilm samples used to generate the results in table 5 were S. mutans specific biofilms.

1. An oral care composition comprising:
a. an amino acid source;
b. a cannabinoid source; and
c. an orally acceptable vehicle.

2. The composition according to claim 1, wherein the amino acid source comprises an amino acid selected from the group consisting of arginine, L-arginine, cysteine, leucine, isoleucine, lysine, L-lysine, alanine, asparagine, aspartate, phenylalanine, glutamate, glutamic acid, threonine, glutamine, tryptophan, glycine, valine, proline, serine, tyrosine, and histidine, and mixtures thereof.

3. The composition according to claim 2, wherein the amino acid is selected from the group consisting of arginine, lysine, glycine and combinations thereof.

4. The composition according to claim 3, wherein the amino acid is arginine in free or salt form.

5. The composition according to claim 4, wherein the amino acid source comprises arginine in an amount from 0.1% to 10% by wt., of the total composition weight, wherein the weight of the arginine is calculated as free form.

6. The composition according to claim 1, wherein the cannabinoid source comprises one or more cannabinoids selected from cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (THCV), Δ⁹-tetrahydrocannabinol (THC), tetrahydrocannabinolic acid (THCA) and combinations thereof.

7. The composition according to claim 1, wherein the cannabinoid is a non-psychoactive cannabinoid.

8. The composition according to claim 1, wherein the cannabinoid source comprises less than 0.3 wt. % Δ⁹-tetrahydrocannabinol (THC) relative to the total weight of the composition.

9. The composition according to claim 1, wherein the cannabinoid source comprises a cannabinoid selected from cannabichromene (CBC), cannabigerol (CBG), cannabidiol (CBD), cannabinol (CBN) and combinations thereof.

10. The composition according to claim 1, wherein the cannabinoid source comprises cannabidiol (CBD).

11. The composition according to claim 1, wherein the cannabinoid source is present in an amount of 0.001 wt. % to 1.0 wt. %, relative to the total weight of the composition.

12. (canceled)

13. The composition according to claim 1, further comprising a fluoride source selected from: sodium fluoride, potassium fluoride, sodium monofluorophosphate, sodium fluorosilicate, ammonium fluorosilicate, amine fluoride, ammonium fluoride, titanium fluoride, hexafluorosulfate, and combinations thereof.

14. A method of controlling bacteria in the oral cavity, the method comprising providing a composition according to claim 1.

15. A method of decreasing mitochondrial respiration and/or glycolysis in an oral biofilm of a subject in need thereof, wherein the method comprises administering the composition of claim 1 to the oral cavity of the subject, and wherein the biofilm comprises S. mutans.

16. The composition of claim 1, wherein the composition comprises:
a. arginine;
b. zinc phosphate;
c. stannous fluoride; and
d. a cannabinoid source comprising cannabidiol.

17. The composition of claim 1, wherein the composition comprises:
a. arginine;
b. zinc citrate and zinc oxide;
c. a cannabinoid source comprising cannabidiol; and
d. sodium fluoride

18. The composition of claim 1, wherein the composition comprises:
a. arginine at about 1.5% by wt.; and
b. a cannabinoid source comprising cannabidiol at about 0.5% by wt, wherein the cannabinoid source comprises Cannabis sativa seed oil (CSO), wherein the CSO further comprises about 5% by wt of cannabidiol (CBD), and wherein the wt. % of CBD is based on the total weight wt. of the CSO.
